(19) United States
(12) Patent Application Publication
(10) Pub. No.: US 2005/0222043 A1
(43) Pub. Date: Oct. 6, 2005

(54) Use of proteasome inhibitor in the treatment of fibrotic diseases

(76) Inventors: Karl Stangl, Berlin (DE); Silke Meiners, Berlin (DE); Berthold Hocher, Kleinmachnow (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/522,370

(22) PCT Filed: Jul. 25, 2003
(86) PCT No.: PCT/EP03/08205

§ 371 Date: May 3, 2005

(30) Foreign Application Priority Data
Jul. 25, 2002 (DE) 102 33 929.5

(51) Int. Cl. A61K 38/08; A61K 38/06; A61K 38/04; A61K 48/00

(52) U.S. Cl. 514/17; 514/18; 514/19; 514/44

(57) ABSTRACT

The invention relates to the use of at least one proteasome inhibitor, preferably in the form of a threonine protease inhibitor, in the treatment of fibrotic diseases, espeially fibrotic diseases of the cardiovascular system.

The present invention relates to the use of at least one proteasome inhibitor for the treatment of fibrotic diseases, especially fibrotic diseases of the cardiovascular system.

Myocardial fibrosis is a reaction to the overload of the myocardium, which may e.g. been caused by high blood pressure, myocardial infarction, or cardiomyopathies. Numerous pathophysiological mechanisms contribute to this phenomenon, like e.g. the proliferation of heart connective tissue cells leading to an increased matrix formation. Functional final states are the mass gain of the myocardium and an increased fibrous reorganisation (fibrosis). One indication of the heart's reorganisation is thus the interstitial fibrosis leading to an increased stiffness of the cardiac walls. This in respect to function leads to a diastolic dysfunction, which may further aggravate the weakness of the myocardium. In part, these effects are caused by an increased expression of genes coding for extracellular matrix proteins like collagen I and III, the dominant collagens of the myocardium.

The currently best established strategy for the reduction of cardiac fibrosis is the pharmacological inhibition of the renin-angiotensin system, e.g. by means of ACE inhibitors or angiotensin II blockers. These substances however have the disadvantage that they will lead to intolerance effects in the patients in at least 5% of the cases. Moreover, they only affect the cardiac fibrosis mediated by the renin-angiotensin-system, but not the development of a myocardial fibrosis mediated by other agonists such as TGF-beta (transforming growth factor beta) or endothelin-1. There is thus the need for alternative methods, which could reduce or prevent a cardiac fibrosis.

It was now surprisingly found, that the inhibition of the proteasome system can effectively reduce or prevent a cardiac fibrosis, thus leading to improved cardiac function.

The subject of the present invention thus is the use of at least one proteasome inhibitor for the treatment of fibrotic diseases. The fibrotic diseases can affect very different organ systems like lung, liver, skin, joints, skeleton and/or glands. In particular, the invention relates to fibrotic diseases of the cardiovascular system.

The ubiquitin-proteasome system is the main metabolic pathway for the decomposition of intracellular proteins in eukaryotic cells, like e.g. signal mediators, cell cycle proteins and transcription factors. It was shown, that an inhibition of the proteasome blocks cellular proliferation, interferes with different signal pathways and affects gene expression (Lee, D. H. et al. (1998) Trends Cell Biol., 8397-403; Yu C.-L. et al. (1997) J. Biol. Chem., 272, 14017-14020; Heldin C.-H. et al. (1999) Nat. Cell Biol., 1, E195-E197 and Desterro, J. M. P. et al., (2000) Cell Mol Live Sci., 57, 1207-1219).

Suitable as proteasome inhibitors are low-molecular organic compounds on the one hand and molecular-biological compounds on the other hand, wherein these substances preferably inhibit the proteasome in a substantially specific manner. The inhibition test can e.g. be performed as being described by Dahlmann B. et al. (2000) J. Mol. Biol., 303, 643-653. The wording “substantially specific” according to the present invention means, that the inhibitor inhibits the proteasome in a more pronounced manner than other intracellular proteases like e.g. calpain, cathepsins or TPPII, wherein this inhibition is preferably about 10 times stronger, in particular is about 100 times stronger, principally is about 1000 times stronger than the inhibition of other intracellular proteases.

The inhibition values are commonly indicated as IC₅₀ (inhibitor concentration at 50% inhibition) and compared with each other. Low-molecular organic compounds according to the present invention refer to organic compounds with a relative molar mass ≦1000, preferably ≦800. Molecular-biological compounds according to the present invention refer to nucleic acids, in particular to RNA or DNA, which inhibit the expression of a component of the proteasomal system, e.g. the transcription or translation of the proteasome encoding nucleic acids, or to proteins, in particular binding peptides or binding proteins, these substances being directed against at least one component of the proteasomal system, preferably against ubiquitin and/or against the proteasome. Said nucleic acid e.g. is an anti-sense-RNA or a double-stranded RNA (dsRNA) against a proteasome encoding sequence, a duplex forming oligonucleotide against a proteasome encoding sequence and/or a knock-out construct against a proteasome encoding sequence. Suitable as binding proteins or binding peptides e.g. are antibodies or their binding-reactive parts, e.g. single chain antibodies (scAb) or Fab-fragments or derivatives thereof, e.g. bi-specific antibodies against at least one component of the proteasomal system. A description of the proteasomal system and of suitable proteasome inhibitors is e.g. found in Kisselev A. F. & Goldberg A. L. (2001) Chemistry & Biology, 8, 739-758.

Suitable examples for proteasome inhibitors thus in particular are threonine protease inhibitors, serine protease inhibitors and/or cysteine protease inhibitors, especially a peptide aldehyde, a peptide boronate, a peptide vinyl sulfone, a peptide epoxyketone, a lactacystine, a peptide alpha keto-aldehyde, an alpha-ketoamide, an indanone peptide, a polyalkylene aldehyde and/or a polyphenol, in particular a cathechin-3-gallate, which can e.g. be extracted from green tea. Especially suitable as proteasome inhibitor are Z-Leu-Leu-Leu-al (MG132), Z-Ile-Glu(OtBu)-Ala-Leu-al (PSI), CEP1612, pyrazylcarbonyl-Phe-Leu-boronate (PS-341), dansyl-Phe-Leu-boronate (DFLB), morpholino-naphthylalanine-Leu-boronate (MG273), NIP-Leu₃-vinylsulfone (NLVS), Tyr-Leu₃-VS, NIP-Leu-Leu-Asn-VS, Ada-Tyr-Ahx₃-Leu₃-VS, Ada-Lys(Bio)-Ahx₃-Leu₃-VS, Ac(Me)-Ile-Ile-Thr-Leu-EX (epoxomicin), dihydroeponemycin, lactacystine, clasto-lactacystine-beta-lactone (omuralide), PS-519, Ac-Leu-Leu-Nle-al (ALLN), 3,4-dichloroisocoumarine (DCI), 4-(2-aminoethyl)-benzenesulfonyl fluoride (Pefablock SC), TMC-95A, gliotoxin, (−)-epigallocatechin-3-gallate (EGCG), ritonavir, lovastatin, aclacinomicin A (Aclarubicin) and/or cyclosporin, which are all described more closely in Kisselev A. F. & Goldberg A. L. (2001, supra) and in FIG. 3, and wherein Z is a benzyloxycarbonyl group, al is an aldehyde group, VS is a vinyl sulfone group, NIP is a 3-nitro-4-hydroxy-5-iodophenylacetate group, and Bio is a biotin group. Particularly preferred of all these classes of compounds are threonine protease inhibitors and, from this, especially the compounds MG132, MG262, lactacystine and/or epoxomicine, above all MG132 and/or MG262.

According to the present invention, the effects the proteasome inhibition exerts on the cardiac fibrosis are studied in spontaneously hypertensive rats (SH-rats). In a long-term treatment of 12 weeks with the specific proteasome inhibitor MG132 at a daily dose of 1 mg per kg we surprisingly found a significant inhibition of the cardiac fibrosis by approximately 40%, moreover showing, that the inhibitor was very well tolerated. The described effects resulted in an improved ventricular function in the MG132-treated animals. What was shown in vitro was a concentration-dependent inhibition of the growth of cardiac fibroblasts and a specific, concentration-dependent down-regulation of collagen Iα2 by approximately 75% and of collagen IIIα1 by approximately 90%. Although one would have expected the systemic treatment with proteasome inhibitors as inhibitors of a crucial cellular system to produce cytotoxicity, the described long-term treatment did not result in measurable side effects, but in contrast was well tolerated. Thus, proteasome inhibitors show a preferable suitability for the treatment of fibrotic diseases, in particular those of the cardiovascular system.

The present invention thus also relates to the treatment of patients suffering from a fibrotic disease, preferably a fibrosis of the cardiovascular system. Possible as forms of application are both the systemic and the local application.

The following methods and results with the corresponding FIGS. 1 to 3 are intended to more closely illustrate the invention in an exemplary manner, without limiting the invention to this.

### DESCRIPTION OF THE FIGURES

FIG. 1 describes the effects of a MG132-treatment of SH-rats on cardiac fibrosis, determined by quantitative morphometry of siriusred-stained, left-ventricular microscopic sections (FIG. 1A-1C) and on cardiac function (FIG. 1D-1F).

FIG. 1A shows a representative section through the heart of an untreated control-SH-rat, which shows an increased myocardial fibrosis. The fibrotic tissue is exemplarily indicated by asterisks. The bar equals 20 μm.

FIG. 1B shows a typical section through the heart of a MG132-treated animal, which shows a significantly reduced cardiac fibrosis under proteasome inhibition.

FIG. 1C shows the quantitative evaluation by means of computer-aided image analysis. The result here is a cardiac fibrosis being reduced by approximately 40% under MG132-treatment. Mean value ±S.E.M. (standard error of mean=standard deviation/root of n), n=7-10, *:p<0,05.

FIG. 1D shows the end-diastolic pressures (LVEDP) in the left ventricle of the rats with a remarkably reduced pressure level in the MG132-treated animals. Mean value ±S.E.M., n=6-10, *:p<0,05, **p<0,01.

FIG. 1E shows the maximal pressure increase rate (dp/dtmax), which is significantly higher in the MG132-treated animals. Mean value ±S.E.M., n=6-10, *:p<0,05, **:p<0,01.

FIG. 1F shows the maximal pressure drop rate (dp/dtmin), which is increased by a factor of 2 under treatment with MG132. Mean value ±S.E.M., n=6-10, *:p<0,05, **:p<0,01.

FIG. 2 shows the effects of proteasome inhibitors on the proliferation and collagen expression in primary cardiac fibroblasts of the rat.

FIG. 2A shows the dose-dependent inhibition of proliferation by MG132. The cells were stimulated with 0,1 μM and 1 μM MG132 or 0,1% DMSO and counted daily. Mean value ±S.E.M., n=3.

FIG. 2B shows the quantification of the mRNA-amount of the collagens Iα1, Iα2 and IIIα1 by means of “real-time” PCR-analysis. Under MG132-treatment, one observes a dose-dependent and dramatic reduction of the expression of the collagens Iα2 and IIIα1. Mean value ±S.E.M., n=2.

FIGS. 3A and B show the chemical structure of different proteasome inhibitors.

FIG. 4 shows a real-time-PCR-analysis of the expression of MMP2 and MMP9 and of the collagens Iα1, Iα2 and IIIα1 both under basal conditions (FIG. 4C or FIG. 4D) and under co-stimulation with MG132 and IL-β (FIG. 4A or FIG. 4B).

FIG. 5 shows zymographic experiments for the detection of active MMP2 and MMP9 under basal conditions (FIG. 5A) or under co-stimulation with proteasome inhibitor and IL-β (FIG. 5B).

FIG. 6 shows a band shift-analysis for the detection of active NFκB in nuclear extracts after stimulation with MG132 and/or with IL-β.

### METHODS

Method 1: Preparation of Cardiac Fibroblasts

Cardiac fibroblasts were prepared from neonatal Wistar rats by plating the non-myocyte fraction of neonatal hearts. The hearts were removed from neonatal rats being 2-3 days old. Ventricular tissue was treated over night at 4° C. with 50 mg/ml trypsin in Hanks-Balanced Salt Saline (HBSS) (Ca²⁺- and Mg²⁺-free, Invitrogen™, Karlsruhe, Germany) and with collagenase (0,5 mg per ml) in Leibowitz medium (L-15, Invitrogen™, Karlsruhe, Germany) for 45 minutes at 37° C. After washing, the cells were pre-plated at 37° C. for one hour. The adherent cells predominantly were cardiac fibroblasts, which were then further cultivated under standard conditions in standard M199-medium with 10% new-born calf serum, L-glutamine and penicillin-streptomycin (Invitrogen™, Karlsruhe, Germany). In the experiments, we used sub-cultivated fibroblasts of the passages 3-7.

Method 2: Proliferation Test

MG132, ALLM and MG262 were purchased at Calbiochem® (San Diego, Calif., USA) and provided as 10 mM DMSO stock solutions. Cardiac fibroblasts (5×10⁴ cells per ml) were inoculated in 24-well-plates. Adherent cells were either stimulated with MG132 (0,1 and 1 μM) or DMSO (0,1%) in medium with 10% serum and further cultivated for 7 days. The medium was exchanged every second day. The proliferation was determined by counting the living cells in triplicate in daily intervals by means of the trypanblue exclusion test. This method allows for the distinction between living and dead cells: the dye trypanblue, which is added to the cells, can only enter into cells having a defective cell membrane, which are consequently stained blue. Living cells with an intact cell membrane are not stained.

Method 3: Histological Determination of Fibrosis

For the pathohistological evaluation, the rat hearts were embedded into paraffin, cut into sections having a thickness of 3 μm, subjected to a haematoxylin-eosin and siriusred staining and analysed as being described in Hocher, B. et al., (1999), Hypertension, 3, 816-822. The degree of heart fibrosis was evaluated by means of quantitative morphometry of the siriusred-stained sections with an image analysis system (Quantimed 500, Image 1.61 program). In this analysis, the ratio of the red-stained tissue (connective tissue) and the total tissue of the section was determined.

Method 4: RNA and RT-PCR-Analysis

Total RNA was extracted by means of Trizol reagent (Invitrogen™, Karlsruhe, Germany). In the following, the RNA was subjected to reverse transcription (AMF Reverse Transkriptase, Roche Biochemicals, Mannheim, Germany) and treated with DNase (Ambion, Huntington, UK). We designed PCR-primers for the rat collagen-RNAs Iα1, Iα2, IIIα1, for the RNAs of the matrix-metalloproteinases (MMP) 2 and 9 and for the “housekeeping” gene hypoxanthine phosphoribosyltransferase (HPRT) by using the PrimerExpress-Software, version 1.2 (PerkinElmer/Applied Biosystems, Wellesley, Mass., USA); the respective primers were then obtained from TIB Molbiol, Berlin, Germany. The PCR reaction was performed with SybrGreen in an ABI PRISM 5700 sequence detector (PerkinElmer/Applied Biosystems, Wellesley, Mass., USA) according to the manufacturer's instructions. The relative quantification was performed by means to the comparative CT-method as being described by the manufacturer.

Method 5: Animal Studies

Male SH-rats of the same age were obtained from the Jackson Laboratory, Maine, USA. The animals were maintained according to the international guidelines for the keeping und use of laboratory animals. 6-week-old rats were treated for 12 weeks with MG132 (1 mg per kg body weight), 0,1% DMSO as solvent control or with physiological saline (n=10 per group, daily intraperitoneal injection). The animals were fed with a high salt diet (drinking water with 2% sodium chloride). The blood pressure was measured weekly by means of the standard tail-cuff method. The blood values and the standard laboratory markers of the renal and hepatic functions were determined 6 weeks after the treatment and at the end of the study by means of standard methods. At the end of the study, the animals were anaesthetised by means of an intraperitoneal injection of a 20% urethane solution (0,9 g per kg body weight) and the left-ventricular pressure parameters were determined as being described by Saragoca, M. et al. (1981) Hypertension, 3, 380-385. The organs were withdrawn, weighed and either shock-frozen in liquid nitrogen or embedded in paraffin for the histological analysis. Since no difference was observed between the DMSO-treated and the salt-treated SH-rats in respect to the above mentioned parameters, the DMSO-treated animals served as a control for the MG132-treatment.

The data was determined in the form of mean values ±S.E.M., if not otherwise indicated. The significance of the differences in the left-ventricular pressure parameters and in the quantification of the heart fibrosis was determined according to Student's T-test. For the cell proliferation test, the significance was determined by comparing the regression coefficients of MG132 in relation to the control group. An error probability of p<0,05 was considered as being significant. The software SPSS 9.0 was used for all statistical calculations.

Results

The treatment of spontaneously hypertensive rats with the specific proteasome inhibitor MG132 for 12 weeks was in general well tolerated during this long period. In blood samples taken after 6 weeks and at the end of the study, neither changes in the differential blood picture nor a change in the laboratory markers, which indicate side effects in the kidney or liver, were observed. Moreover, the MG132-treated animals showed no significant change in the systolic blood pressure and in the heart weight (table 1).

TABLE 1

| | Control | 1 mg/kg MG132 |
|---|---|---|
| Systolic BD (mm Hg) | 196.75 ± 9.1 | 191.43 ± 11.2 |
| Body Weight (g) | 286 ± 36.6 | 280 ± 15.3 |
| Heart Weight (g) | 1.28 ± 0.07 | 1.18 ± 0.07 |
| HG/KG (mg/g) | 4.64 ± 0.61 | 4.1 ± 0.34 |

BD means blood pressure; KG means body weight and HG means heart weight.

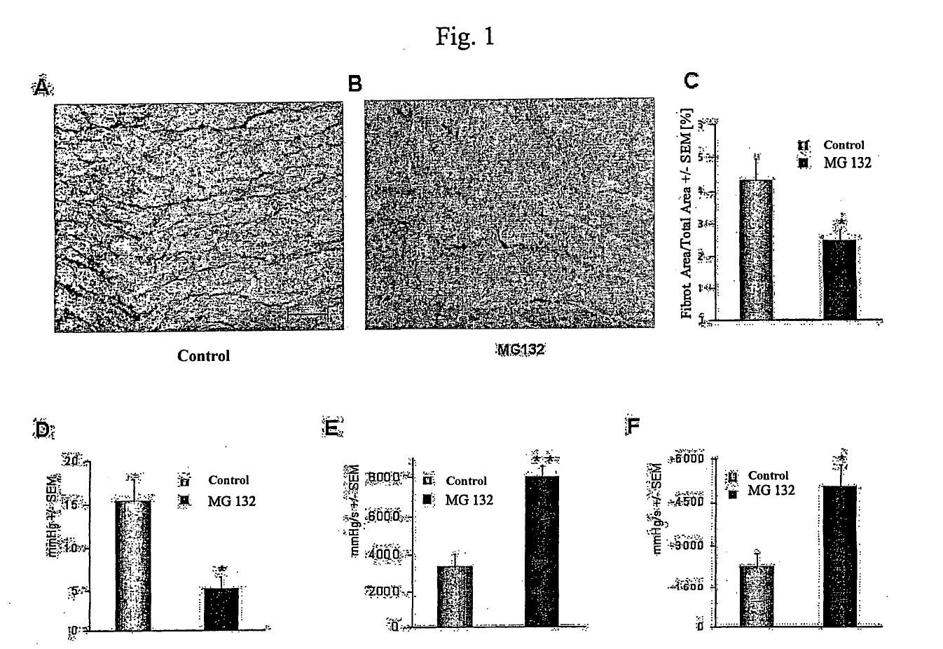

As it can be seen in FIG. 1A to 1C, the MG132-treated SH-rats showed a significant reduction of the heart fibrosis (−38%) in comparison to the control animals, when determining the results by means of quantitative morphometry of siriusred-stained sections of the left ventricles.

The prevention of cardiac fibrosis in MG132-treated SH-rats correlated well with the normal left-ventricular function, whereas the controls showed indications of a beginning left-ventricular dysfunction as shown in the FIGS. 1D-1F: The filling pressures (LVEDP) were significantly lower (FIG. 1D: 15±2 versus 5±3 mm Hg, p=0,017), the maximal pressure increase rate dp/dtmax (FIG. 1E: 8010±538 versus 3375±662 mm Hg, p=0,003) and the maximal pressure drop rate dp/dtmin (FIG. 1F: −5046±726 versus −2290±422 mm Hg/s, p=0,015)—which are parameters of cardiac inotropy and cardiac lusitropy—were more than twice higher in the MG132-treated SH-rats in comparison to the control SH-rats.

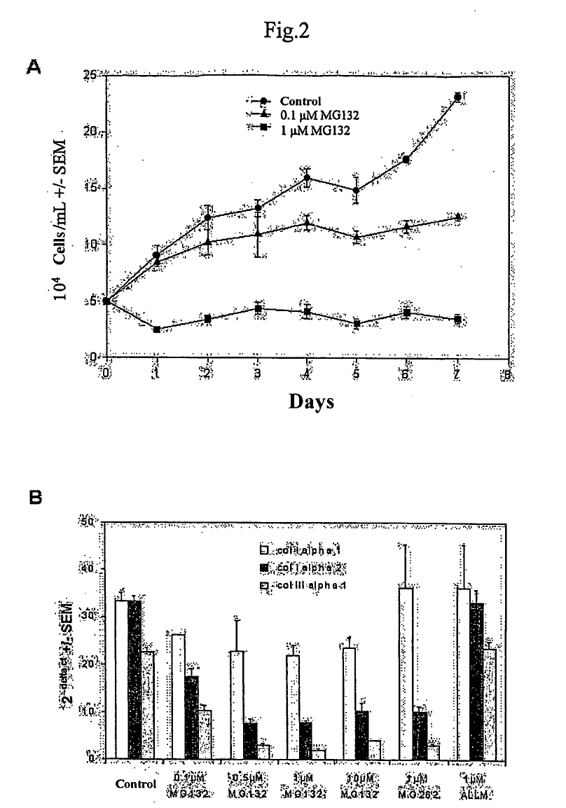

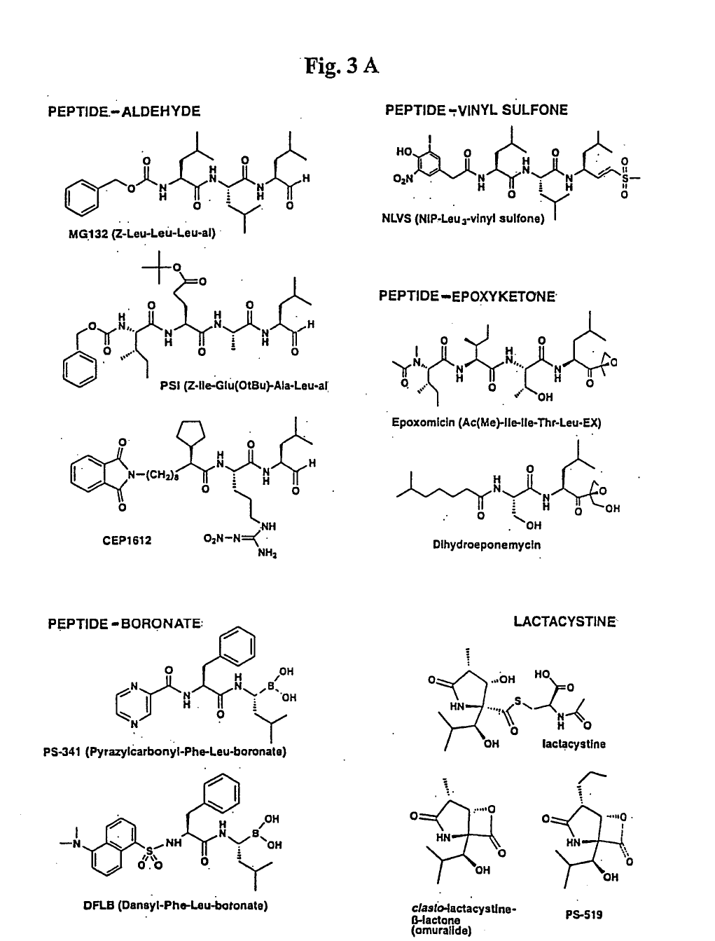

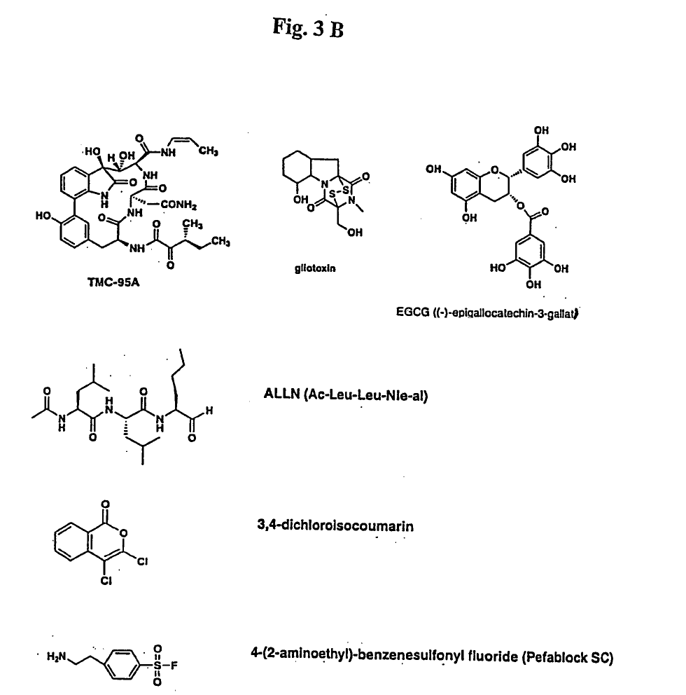

In order to elucidate possible mechanisms, by which the proteasome inhibition may contribute in vivo to the reduced cardiac fibrosis, primary cardiac fibroblasts of the rat were treated with proteasome inhibitors. As illustrated in FIG. 2A, the treatment of cardiac fibroblasts with 0,1 and 1 μM MG132 induced a dose-dependent inhibition of proliferation. Moreover, the RNA-expression of collagen Iα2 and IIIα1 was inhibited by MG132 in a dose-dependent manner by up to 73% and up to 91% (FIG. 2B). In contrast to this, the expression of collagen Ial was unaffected. A second specific proteasome inhibitor, MG262, which is a boronate derivative of MG132, inhibited the collagen expression in a comparably effective manner (FIG. 2B) and thus proves the specificity of the proteasomal inhibition. The cathepsin inhibitor ALLM, which as a peptide aldehyde (ALLM-al) is structurally related to MG132, in contrast showed no effects on collagen expression (FIG. 2B).

The endemically occurring and—in respects of health policy—extremely important organ fibroses (especially myocardial fibrosis) have to be completely distinguished from extreme variants of fibrotic processes in the form of inflammatory responses to foreign matters (see also capsule formation in case of silicone implants), which are reported about by H. Rupp, P. Newrzella, H. König and B. Maisch in: “Hemmung der kardialen Fibrose durch Blockierung des Transkriptionsfaktors NF-kappaB” at the 67^{th} annual meeting of the “Deutsche Gesellschaft für Kardiologie-Herz-und Kreislaufforschung, Apr. 19-21, 2001, Mannheim.

Especially in the case of the myocardial fibrosis which occurs million fold and is—according to the invention—treatable by the application of proteasome inhibitors for prevention or therapy, the pathophysiological agent is the neuroendocrinological activation with the release of vasoconstricting mediators like cathecholamins, angiotensin II, endothelin-1, TGF-beta and other factors, which are released in an increased manner under the conditions of a systemic overload (e.g. a chronic pressure stress in case of arterial hypertension) or a regional overload (e.g. compensatory hyperkinesis of the intact residual myocardium in case of a myocardial infarction). Besides their vasoconstricting properties, these mediators have strong growth inducing effects in the sense of a mass gain of heart muscle cells and the proliferation and increased synthesis output in the form of extracellular matrix formation of cardiac fibroblasts.

In case of fibrotic hearts damaged by overload and, according to the invention, treated with proteasome inhibitors, normally no indication for an inflammatory reaction is observed in the exact tissue analysis. It is scientifically well proved that said cardiac fibroses being induced by overload are independent from the activation of the inflammatory transcription factor NFκB and follow the signal pathway of TGF-beta (see Kitamoto et al., Circulation 2000; 102:806-12; Lijnen et al., Mol Gen Metab 2000; 71:418-35). The fibrosis of cardiovascular relevance which according to the invention is treatable by proteasome inhibitors is thus caused independently from NFκB by different forms of overload with consecutive neuroendocrinological activation.

These positions are clearly supported thereby, that the main substance groups being directed against the development of a fibrosis or contributing to the regression of a fibrosis have antagonistic effects on specific vasoactive mediators. Good examples for this are angiotensin converting enzyme inhibitors (ACE inhibitors), angiotensin II type 1-receptor antagonists (AT-1 antagonists) and endothelin receptor antagonists.

Other preferred forms of organ fibroses without a dominating inflammatory component, which according to the invention are treatable by proteasome inhibitors are the liver fibrosis caused by congestion, the kidney fibrosis caused by high pressure and joint fibroses in case of malpositions.

Experimental data of the present invention verify that the inventive anti-fibrotic effects of proteasome inhibitors are independent from the activation of the inflammatory transcription factor NFκB.

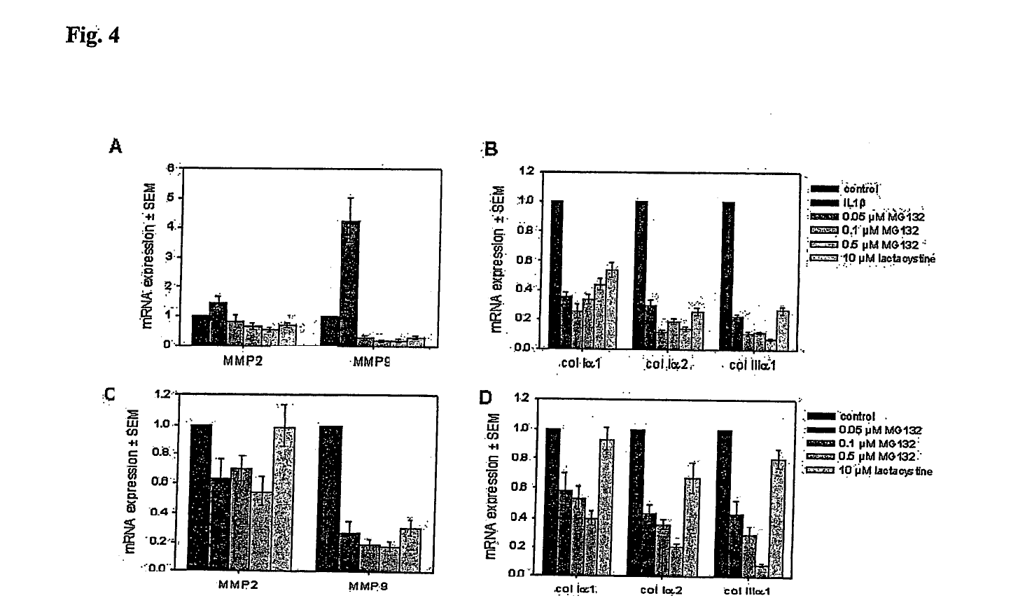

1. Real-Time PCR-Analysis of the Expression of Collagens and MMP2 and 9 (FIG. 4):

For this analysis, cardiac fibroblasts were treated with proteasome inhibitors for 24 hours under serum-free conditions. The prepared RNA was then used to determine the expression of the collagens Iα1, Iα2 und IIIα1 and of MMP2 and MMP9 by means of real-time (RT)-PCR.

As it is shown in the FIGS. 4C and 4D, the inventive inhibition of the proteasome not only significantly inhibited the expression of the collagens, but also the expression of the MMPs. This inventive inhibitory effect of the proteasome inhibitors was observed under basal conditions and without an additional cytokine stimulation of the cells.

In order to investigate, if the proteasome inhibitors also show these effects after the induction of the MMPs caused by stimulation with the NFκB-activating cytokine interleukin-1β (IL-1β), the cardiac fibroblasts were co-stimulated with IL-1β and proteasome inhibitor.

The IL-1β-stimulation led to a significant increase of the expression of the MMPs, especially of MMP9 (FIG. 4A). This MMP has been described as a protease, which can be activated by NFκB and stimulated by cytokine treatment (Gum et al., J Biol. Chem. 1996; 271:10672-80). The simultaneous administration of proteasome inhibitors not only prevented the IL-1β-induced increase of expression, but surprisingly reduced the expression to less than the DMSO control values (FIG. 4A). This demonstrates the inventive inhibition of the MMP-expression by means of proteasome inhibition, which leads to an inhibition of the MMP-expression being stronger than the inhibition of the IL-1β mediated MMP-expression, thus being a process independent from inhibiting the activation of NFκB.

The inventive effect of the proteasome inhibition being independent from NFκB-inhibition in the inventive prevention and treatment of fibrotic diseases was demonstrated even more impressively in an analysis of collagen expression under IL-1β stimulation (FIG. 4B).

As already being described by Siwik et al., (Siwik et al., Am J Physiol Cell Physiol. 2001; 280:C53-C60), the IL-1β treatment led to a down-regulation of the collagen expression in cardiac fibroblasts (FIG. 4B). This suppression can be explained by down-regulating NFκB-elements in the promoter of the collagens, i.e. by a suppressing effect of activated NFκB on collagen expression (Kouba et al., J. Immunol. 1999; 162:3226-34). The co-stimulation of the fibroblasts with IL-1β and with proteasome inhibitors however did not result in a reversal of this suppression of the collagens by NFκB as it would have been expected in an inhibition of the activation of NFκB by proteasome inhibitors, but surprisingly led to an even stronger suppression of the collagen expression than achieved with IL-1β alone (FIG. 4B).

The present investigations demonstrate that the inventive treatment of fibrotic diseases by means of proteasome inhibitors is independent from the inhibition of the activation of NFκB. Advantageously, the invention allows for the inhibition of the expression of MMPs and collagens in fibrotic diseases in a manner being independent from the inhibition of the activation of NFκB. Preferably, the invention enables the prevention and therapy of fibrotic diseases, which are not or not predominantly mediated by NFκB.

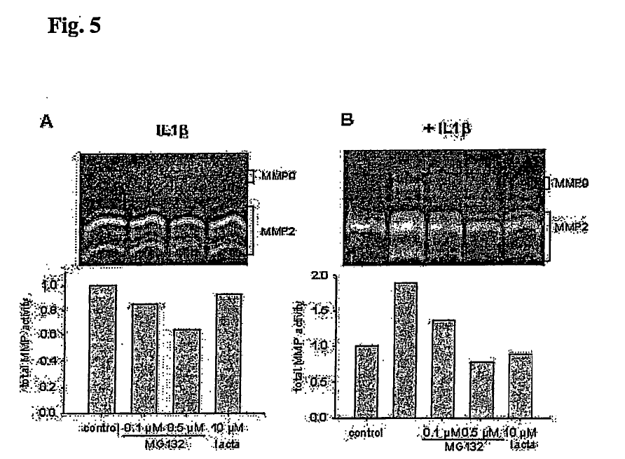

2. Zymography for the Detection of Active MMP2 and MMP9 (FIG. 5):

The inventive inhibition of the expression of active MMP2 in cardiac fibroblasts under basal, i.e. not NFκB-stimulated conditions by means of proteasome inhibitors was also verified in zymography experiments (FIG. 5). In these experiments, cell culture supernatants were investigated after the incubation with proteasome inhibitors in the absence (FIG. 5A) and presence (FIG. 5B) of IL-1β in respect to their gelatinase activity by means of zymography.

According to the invention, the MMP2-formation is already reduced by the proteasome inhibitor in the absence of Il-1β, i.e. in a manner being independent from NFκB (FIG. 5A). The IL-1β-induced MMP2- and MMP9-activation was also reduced by the simultaneous administration of proteasome inhibitors (FIG. 5B).

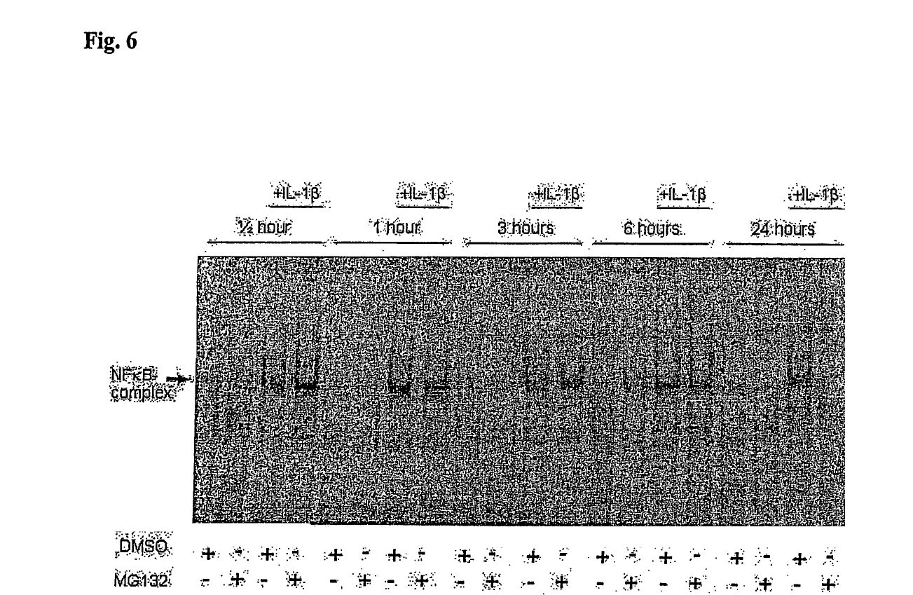

3. Band Shift Analysis for the Detection of Active NFκB (FIG. 6):

The above presented results show that the inventive inhibitory effects of the proteasome inhibitors on collagen- and MMP-expression take place in non-stimulated cardiac fibroblasts and are thus independent from the inhibition of NFκB activation. This was additionally confirmed in band shift analyses with oligonucleotides comprising a NFκB-DNA binding site. For this test, nuclear extracts from cardiac fibroblasts were prepared, which were treated with 0,5 μM MG132 or, respectively, with DMSO (solvent control) in a time course in the presence or absence of IL-1β.

As shown in FIG. 6, cardiac fibroblasts have no active NFκB complex under non-stimulated, basal conditions (see control bands). Under IL-1β, stimulation, an activation of NFκB takes place, represented as band shift. The simultaneous treatment with 0,5 μM MG132 took not less than 6 hours to lead to a small inhibitory effect on NFκB, which however is in no way sufficient to explain the surprisingly strong reduction of the expression of MMP2 and MMP9 in case of the simultaneous application of MG132 and IL-1β (see above, FIG. 4A). After 24 h, one observed a decrease of the IL-1β-induced activation of NFκB with and without MG132. The in vitro experiments thus show that the inventive inhibitory effects of proteasome inhibitors on the expression of collagens and MMPs are mediated independently from the inflammatory transcription factor NFκB.

Also the data presented at the beginning for the inventive prevention of cardiac fibrosis in the animal model of the spontaneously hypertensive rats give evidence for the inventive, MG132-mediated suppression of a collagen expression being independent of inflammatory stimuli. This animal model, according to the invention, does not see an inflammatory stimulus as the trigger for cardiac fibrosis, but a pronounced hypertonia of the rats.

According to the invention, proteasome inhibitors do not only interfere in an inhibitory manner with collagen- and MMP-expression, but display their broad spectrum of activity also in respect to the suppression of fibroblast proliferation.

The inventive therapeutic interventions preferably comprise the systemic application of proteasome inhibitors. In this approach, preferably at least one proteasome inhibitor is administered to the patient in a dose, in which systemic side effects, in particular cytotoxic side effects, of proteasome inhibitors are advantageously avoided or occur only to a small degree. The inventive use of proteasome inhibitors for the prevention and therapy of fibroses is thus well tolerated, preferably allows for a specific anti-fibrotic treatment and therefore enables an inventive prevention and therapy in a variety of patients.

In preferred embodiments, an inventive systemic application of at least one proteasome inhibitor enables the prevention and treatment preferably of a cardiac fibrosis, preferably of a cardiac fibrosis caused by overload, preferably of a cardiac fibrosis caused by overload under chronic pressure stress in arterial hypertension, preferably of a cardiac fibrosis caused by overload in compensatory hyperkinesia of the intact residual myocardium in case of myocardial infarction, preferably of a cardiac fibrosis caused by overload with consecutive neuroendocrinological activation, preferably of a cardiac fibrosis, in case of which a treatment with ACE inhibitors, AT-1-antagonists and/or endothelin receptor antagonists is indicated, preferably of a liver fibrosis caused by congestion, preferably of a kidney fibrosis caused by high pressure and preferably of all joint fibroses in case of joint malpositions.

In the prevention and therapy of fibroses according to the invention, a patient is given at least one proteasome inhibitor, preferably in a dose of approximately 0,5 μg/kg body weight to approximately 0,5 mg/kg body weight, preferably in a dose of approximately 1 μg/kg body weight to approximately 0,1 mg/kg body weight, preferably in a dose of approximately 0,01 mg/kg body weight to approximately 0,1 mg/kg body weight. These doses refer to all of the proteasome inhibitors mentioned in this specification, especially to the threonine protease inhibitors, in particular to MG132 and MG262. Preferably, at least one of the proteasome inhibitors mentioned at the beginning of this specification is administered, preferably MG132. For this aim, one preferably uses advantageous pharmaceutical formulations, which are familiar to the expert, preferably solid and liquid medicinal preparations, preferably solutions for infusion, which preferably contain at least one advantageous pharmaceutical additive, which is known to the expert and which contributes to the shelf life and/or the reduction of side effects of the medical composition.

1. Use of at least one proteasome inhibitor for the treatment of fibrotic diseases, which are not caused by inflammatory responses to foreign matters.

2. Use according to claim 1 for the treatment of a cardiac fibrosis caused by overload, a liver fibrosis caused by congestion, a kidney fibrosis caused by high pressure or a joint fibrosis in case of a joint malposition.

3. Use according to claim 2 for the treatment of a cardiac fibrosis caused by overload under chronic pressure stress in arterial hypertension and/or for the treatment of a cardiac fibrosis caused by overload in compensatory hyperkinesia of the intact residual myocardium in case of myocardial infarction.

4. Use according to claim 2 for the treatment of a cardiac fibrosis, in which a treatment with ACE inhibitors, AT-1-antagonists and/or endothelin receptor antagonists is indicated.

5. Use according to claim 1, wherein a patient is administered at least one proteasome inhibitor in a dose of approximately 0,5 μg/kg body weight to approximately 0,5 mg/kg body weight, preferably in a dose of approximately 1 μg/kg body weight to approximately 0,1 mg/kg body weight, preferably in a dose of approximately 0,01 mg/kg body weight to approximately 0,1 mg/kg body weight.

6. Use according to claim 1, characterised in that the fibrotic diseases relate to fibrotic organ diseases, preferably of the lung, liver, skin, joints, skeleton and/or glands, in particular to diseases of the cardiovascular system.

7. Use according to claim 1, characterised in that the proteasome inhibitor is a low-molecular organic compound or a molecular-biological compound.

8. Use according to claim 7, characterised in that the proteasome inhibitor is a threonine protease inhibitor, a serine protease inhibitor, a cysteine protease inhibitor, a gene expression inhibitor of the proteasomal system and/or a binding protein or binding peptide directed against at least one component of the proteasomal system, preferably against ubiquitin and/or against the proteasome.

9. Use according to claim 7, characterised in that the proteasome inhibitor is a peptide aldehyde, a peptide boronate, a peptide vinyl sulfone, a peptide epoxyketone, a lactacystine, a peptide alpha keto-aldehyde, an alpha-ketoamide, an indanone peptide, a polyalkylene aldehyde, a polyphenol, in particular a cathechin-3-gallate, a nucleic acid directed against at least one component of the proteasomal system and/or an antibody or binding-reactive part or derivative thereof, directed against at least one component of the proteasomal system.

10. Use according to claim 7, characterised in that the proteasome inhibitor is Z-Leu-Leu-Leu-al (MG132), Z-Ile-Glu(OtBu)-Ala-Leu-al (PSI), CEP1612, pyrazylcarbonyl-Phe-Leu-boronate (PS-341), dansyl-Phe-Leu-boronate (DFLB), morpholino-naphthylalanine-Leu-boronate (MG273), NIP-Leu₃-vinylsulfone (NLVS), Tyr-Leu₃-VS, NIP-Leu-Leu-Asn-VS, Ada-Tyr-Ahx₃-Leu₃-VS, Ada-Lys(Bio)-Ahx₃-Leu₃-VS, Ac(Me)-Ile-Ile-Thr-Leu-EX (epoxomicin), dihydroeponemycin, lactacystine, clasto-lactacystine-beta-lactone (omuralide), PS-519, Ac-Leu-Leu-Nle-al (ALLN), 3,4-dichloroisocoumarine (DCI), 4-(2-aminoethyl)-benzenesulfonyl fluoride (Pefablock SC), TMC-95A, gliotoxin, (−)-epigallocatechin-3-gallate (EGCG), ritonavir, lovastatin, aclacinomicin A (Aclarubicin), cyclosporin, an anti-sense-RNA or a double-stranded RNA (dsRNA) against a proteasome encoding sequence, a triplex forming oligonucleotide against a proteasome encoding sequence and/or a knock-out construct against a proteasome encoding sequence, wherein Z is a benzyloxycarbonyl group, al is an aldehyde group, VS is a vinyl sulfone group, NIP is a 3-nitro-4-hydroxy-5-iodophenylacetate group, and Bio is a biotin group.
